(19) United States
(12) Patent Application Publication
(10) Pub. No.: US 2010/0155173 A1
(43) Pub. Date: Jun. 24, 2010

(54) Stethoscope with built-in light

(76) Inventors: Stacy L. Boyd, Taft, CA (US); Travis D. Neumann, Taft, CA (US)

Correspondence Address: KLEIN, DENATALE, GOLDNER, COOPER et. al., P.O. BOX 11172, BAKERSFIELD, CA, 93389-1172, US

(21) Appl. No.: 12/384,334

(22) Filed: Apr. 1, 2009

Related U.S. Application Data
(60) Provisional application No. 61/139,986, filed on Dec. 22, 2008.

(51) Int. Cl. A61B 7/02

(52) U.S. Cl. 181/131; 181/141

(57) ABSTRACT

A stethoscope comprises a pair of binaurals, an acoustical tubing and a chestpiece. There may be a light source within the chestpiece suitable for medical examination purposes. The electrical supply means for operating the light source may comprise one or more batteries. The chestpiece comprises an upper member and a lower member, which may be releasably attached. The chestpiece may have a contoured gripping surface. The light source may have switching means that may be pushed and held to activate the light, and released to deactivate the light, or may be pushed to activate the light, and pushed again to deactivate the light. The acoustical tubing may have measuring indicia that may comprise a pupil dilation measuring scale or a scaled ruler.

### CROSS-REFERENCE TO RELATED APPLICATION

U.S. Provisional Application No. 61/139,986 for this invention was filed on Dec. 22, 2008, for which application these inventors claim domestic priority.

### BACKGROUND

The present invention relates to a stethoscope having a light suitable for patient examination and having measuring indicia along the acoustical tubing. More particularly, the invention relates to a stethoscope providing a plurality of measuring indicia along the acoustical tubing and providing a narrow beam light source where the light source emits light from the side of the stethoscope chestpiece opposite the point where the acoustic tube enters the chestpiece. In this manner a medical practitioner always has a convenient source of light within arm's reach suitable for patient examination. The measuring indicia may comprise a pupil size chart, a measured ruler, or a measurement equivalency chart. The light source may comprise a light-emitting diode or incandescent bulb.

A small but focused beam of light is frequently used by medical practitioners for examining patients' eyes, ears, nose and throat, among other uses. For example, physicians and nurses use light beams to examine patients' eyes, to chart pupil size and response to light, as well as to inspect portions and alignment of the eyes and characteristics of the external eye structure. As the measuring indicia may comprise a pupil size chart, the accuracy of measurement of pupil size is increased as the medical practitioner has a handy nearby reference, rather than having to resort to an approximation of pupil size. With regard to the nose and sinus, light is useful in determining source or nature of sinus drainage, or detect nasal obstruction that may interfere with breathing. Medical practitioners normally use small, individual flashlights to provide light sources, however these are not always readily at hand or might be misplaced.

Accordingly, it would be desirable to have a light source in combination with a stethoscope having measuring indicia. Stethoscopes are generally worn about the neck, even when not in use. Because of their indispensability and frequent use, stethoscopes are seldom, if ever, misplaced. By having a light available at arm's reach, and the measuring indicia, the medical practitioner is provided with frequently used diagnostic aides readily at hand.

### SUMMARY

In accordance with an embodiment of the present invention, there is provided a stethoscope having a light source in its chestpiece. The stethoscope generally comprises a pair of binaurals connected at a junction to a first end of an acoustical tubing which leads to and connects at a second end to the chestpiece. The acoustical tubing may have measuring indicia to assist the user in measuring such things as pupil dilation, the size of an injury or skin feature, etc., wherein the measuring indicia relieves the users from having to approximate the relative size of the subject of interest, or to carry additional measuring devices.

The chestpiece of one embodiment comprises an upper member releasably attached to a lower member. The upper member may be non-metallic. The lower member may be metallic, and the lower member comprises a conical bell and a diaphragm. The chestpiece may comprise a light source which is disposed at the front end of the chestpiece, and the light source may be disposed in the upper member, in the lower member, or in both. The light source comprises a light bulb, a source of power such as a battery, and means for activating or deactivating the electrical connection between the light source and the bulb.

### BRIEF DESCRIPTION OF THE DRAWINGS

FIG. 1 shows a perspective view of an embodiment of the stethoscope with built-in light and measuring indicia.

FIG. 2 shows a perspective view of an embodiment of the chestpiece.

FIG. 3 shows another perspective view of the embodiment of the chestpiece shown in FIG. 2.

FIG. 4 shows an exploded view of the chestpiece shown in FIG. 2.

FIG. 5 shows a top view of the chestpiece shown in FIG. 2.

FIG. 6 is a side view of the embodiment of the chestpiece shown in FIG. 2.

FIG. 7 is a front view of the chestpiece shown in FIG. 2.

FIG. 8 is a cross-sectional view of the chestpiece, taken essentially along the lines 8-8 of FIG. 7.

FIG. 9 is an embodiment of the measuring indicia of the acoustical tubing, showing a measuring scale.

FIG. 10 is an alternate embodiment of the measuring indicia, and shows a pupil dilation chart.

### DETAILED DESCRIPTION OF THE EMBODIMENTS

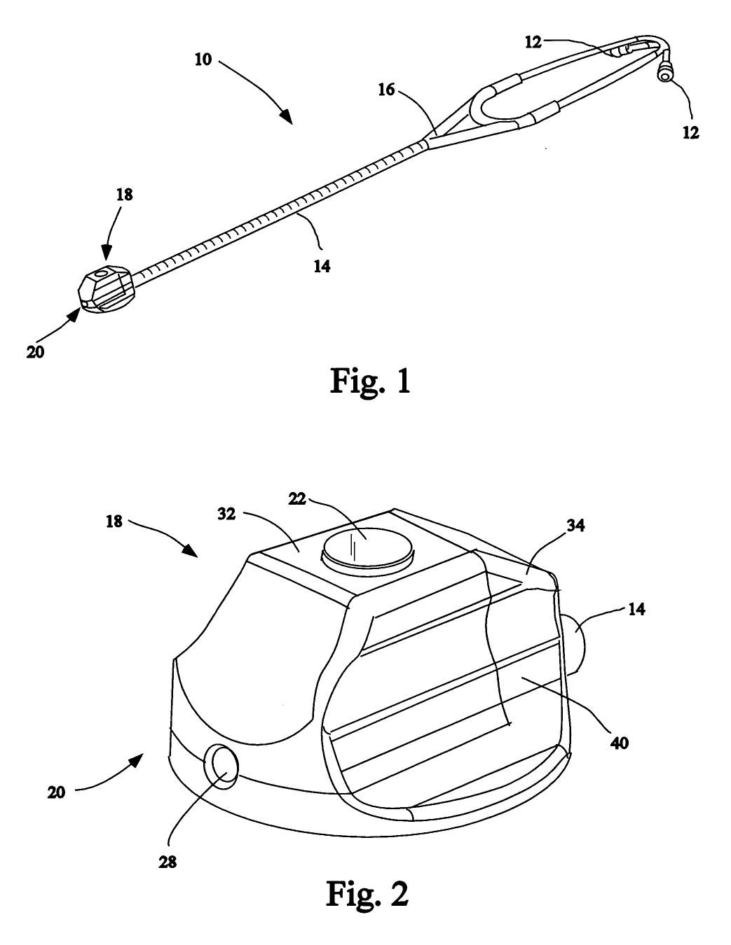

With reference to the Figures, FIG. 1 shows an embodiment of the disclosed stethoscope 10. The stethoscope 10 comprises a pair of binaurals 12 joined through acoustical tubing 14 to a chestpiece 18. The acoustical tubing 14 connects to the binaurals 12 at a junction 16 which links the binaurals 12.

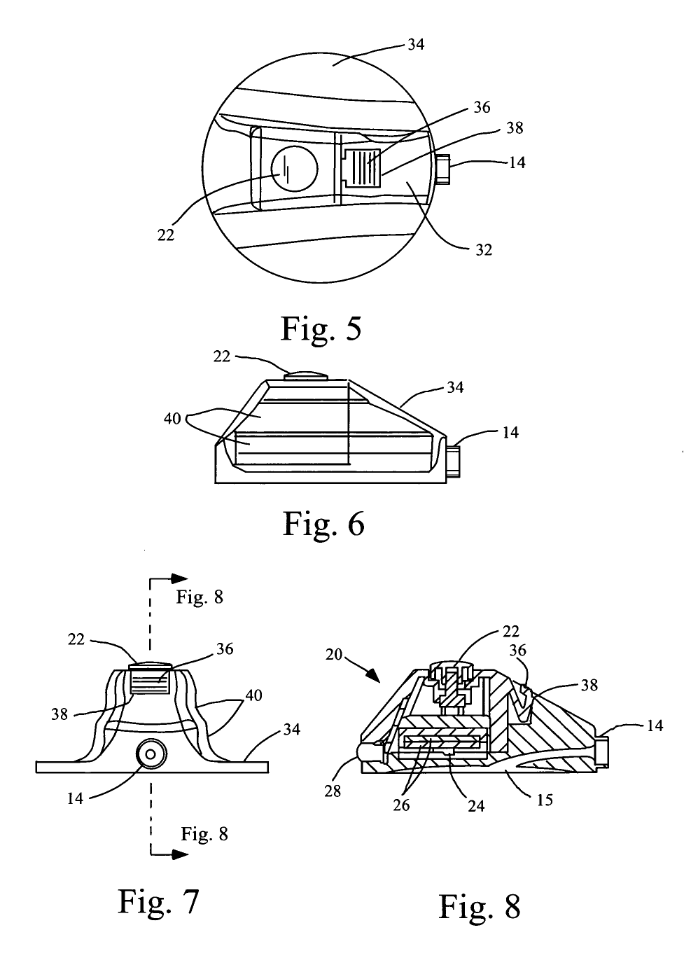

The chestpiece 18 is shown in more detail in FIGS. 2 through 7. The chestpiece 18 is connected to the acoustical tubing 14 and transmits sounds from the patient being examined to the medical practitioner via a diaphragm 15 disposed within the chestpiece 18. The chestpiece 18 may comprise a light source 20 that could be used during a medical examination. The light source 20 may comprise a switching means 22, a metallic contact 24, one or more batteries 26, and a bulb 28. The switching means 22 may be pressed to activate the light source 20 and released to deactivate the light source 20. Alternatively, the switching means 22 may be pressed to activate the light source 20 and pressed again to deactivate the light source 20. The bulb 28 may comprise a light-emitting diode or an incandescent bulb, depending upon the needs of the medical practitioner. Referring to FIG. 8, the light source 20 is shown in the off position, as the metallic contact 24 is not in contact with the lower member 34. The metallic contact 24 contacts the lower member 34 when the switching means 22 is activated, thereby completing the electrical circuit between the light source 20, the switching means 22, and the battery 26, thus providing electrical power to the bulb 28, and the bulb 28 is then illuminated. The bulb 28 may comprise a light-emitting diode which could emit white light, although other colors of LEDs may be chosen if desired for differing medical examination purposes. Alternatively, the bulb 28 may comprise an incandescent bulb.

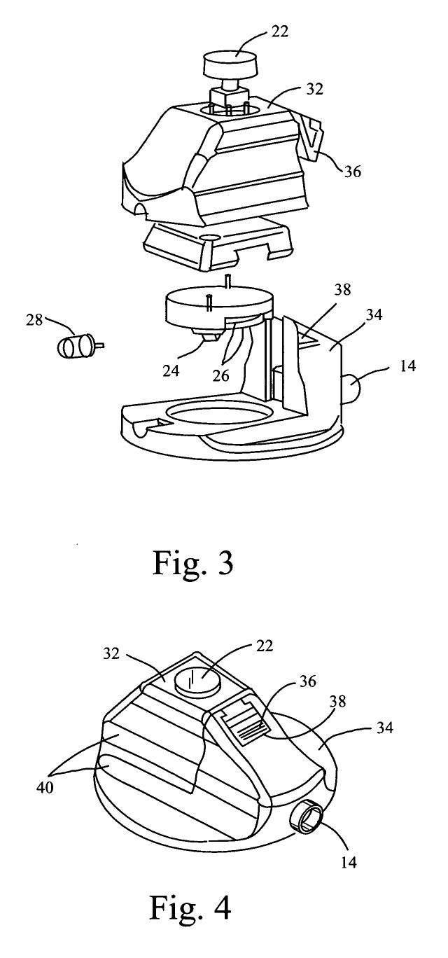

The chestpiece 18 may further comprise an upper member 32 and a lower member 34, and the upper member 32 and the lower member 34 may be releasably attached to one another. The lower member 34 is the portion of the chestpiece 18 that is placed in contact with the patient during the medical examination, and the upper member 32 is the portion of the chestpiece 18 which will generally be held by the medical practitioner. The upper member 32 may be used to house the light source 20 or the lower member 34 may be used to house the lights source 20. Alternatively, as shown in FIG. 4, both the upper member 32 and the lower member 34 may be used to house the lights source 20. The lower member 34 may be used to house the diaphragm and bell of the stethoscope. The lower member 34 may be formed from a metal. The releasable attachment of upper member 32 and lower member 34 facilitates changing bulbs 28 and batteries 26 as these items reach the end of their duty cycle. As best shown in FIG. 3, a release mechanism may be utilized, with a biasing member 36 engaged in a slot 38, thereby retaining the upper member 32 to the lower member 34. It is to be appreciated that the shown releasable closure of the upper member 32 to the lower member 34 is one of several embodiments which might be used.

A contoured gripping surface 40 may be disposed upon the chestpiece 18 to facilitate gripping and holding the chestpiece 18. The contoured gripping surface 40 may extend along both the upper member 32 and the lower member 34, or may occur only along the upper member 32 or only along the lower member 34.

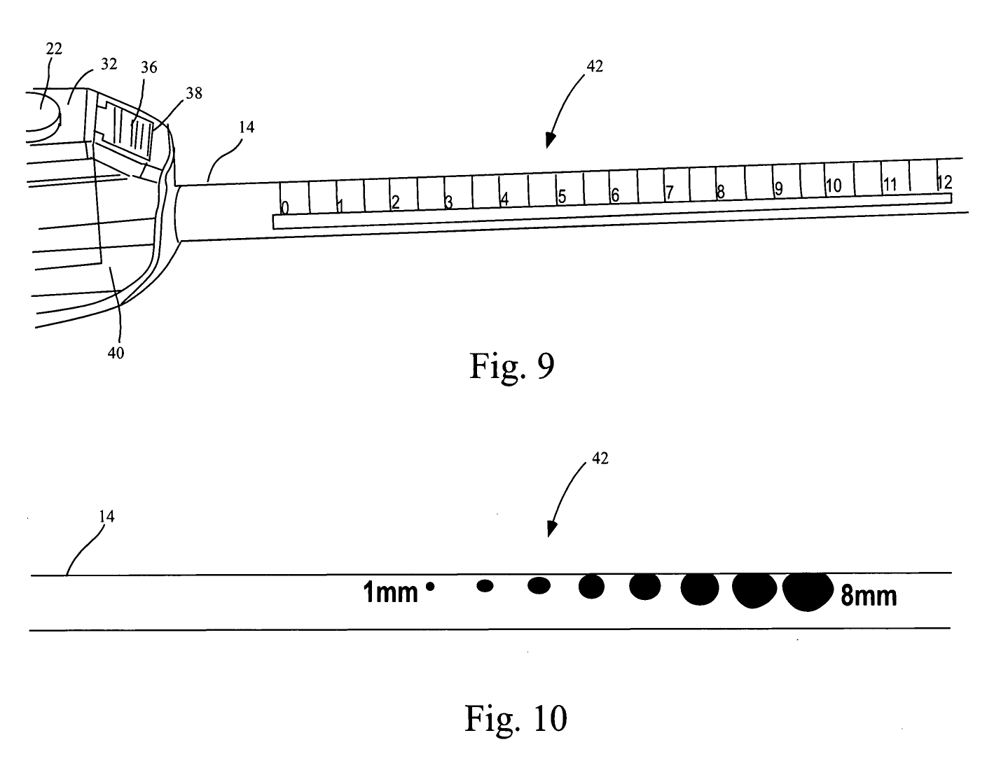

FIGS. 9 and 10 show differing embodiments of a measuring indicia 42 which may be disposed on the acoustical tubing 14. FIG. 9 shows the measuring indicia 42 as a measuring scale. The measuring indicia 42 may be oriented in ascending order from left to right as shown, or may be in descending order from left to right, based upon preference. FIG. 10 is an alternate embodiment of the measuring indicia 42 and shows a pupil dilation chart. Alternatively, the acoustical tubing may comprise as measuring indicia both a pupil dilation chart and a measuring scale as measuring indicia 42.

While the above is a description of various embodiments of the present invention, further modifications may be employed without departing from the spirit and scope of the present invention. Thus the scope of the invention should not be limited by the specific structures disclosed. Instead the true scope of the invention should be determined by the appended claims.

What is claimed is:

1. A stethoscope comprising;
a pair of binaurals;
an acoustical tubing having a first end connected to said binaurals at a junction, and a second end;
a chestpiece attached to said acoustical tubing second end, said chestpiece being capable of receiving internal sounds from a patient;
a light source within said chestpiece, wherein said light source provides a beam of light suitable for medical examination purposes.

2. The stethoscope of claim 1 wherein said light source comprises one or more batteries.

3. The stethoscope of claim 1 wherein said light source comprises a light-emitting diode.

4. The stethoscope of claim 1 wherein said light source comprises an incandescent bulb.

5. The stethoscope of claim 1 wherein said chestpiece comprises an upper member and a lower member, wherein said upper member and said lower member are releasably attached.

6. The stethoscope of claim 5 wherein said upper member is non-metallic and said lower member is metallic.

7. The stethoscope of claim 1 further comprising switching means located in said chestpiece for activating and deactivating said light source.

8. The stethoscope of claim 7 wherein said switching means is a button that is pushed and held to activate said light, and released to deactivate said light.

9. The stethoscope of claim 7 wherein said switching means is a button that is pushed to activate said light, and pushed again to deactivate said light.

10. The stethoscope of claim 1 wherein said acoustical tubing further comprises measuring indicia.

11. The stethoscope of claim 10 wherein said measuring indicia comprise a pupil dilation measuring scale.

12. The stethoscope of claim 10 wherein said measuring indicia comprise a scaled ruler.

13. The stethoscope of claim 12 wherein said scaled ruler comprises a metric scale.

14. A stethoscope comprising;
a pair of binaurals;
an acoustical tubing having a first end and a second end, said first end connected to said binaurals at a junction, wherein said acoustical tubing comprises measuring indicia;
a chestpiece attached to said second end of said acoustical tubing, said chestpiece being capable of receiving internal sounds from a patient.

15. The stethoscope of claim 14 wherein said measuring indicia comprises a pupil dilation measuring scale.

16. The stethoscope of claim 14 wherein said measuring indicia comprise a metric ruler.

17. The stethoscope of claim 14 wherein said chestpiece further comprises a light source within said chestpiece, wherein said light source provides a beam of light suitable for medical examination purposes.

18. An acoustical tubing comprising:
a first end adapted to be connected to a pair of binaurals at a junction and a second end adapted to be attached to a chestpiece, said acoustical tubing comprising measuring indicia.

19. The acoustical tubing of claim 18 wherein said measuring indicia comprise a pupil dilation measuring scale.

20. The acoustical tubing of claim 18 wherein said measuring indicia comprise a scaled ruler.
